(19) United States
(12) Patent Application Publication
(10) Pub. No.: US 2003/0133908 A1
(43) Pub. Date: Jul. 17, 2003

(54) Treatment for intracranial tumors

(76) Inventor: Wolfgang J. Streit, Alachua, FL (US)

Correspondence Address: Stanley A. Kim, Ph., D. , Esq., Akerman Senterfitt, Suite 400, 222 Lakeview Avenue, West Palm Beach, FL, 33402-3188, US

(21) Appl. No.: 10/340,284

(22) Filed: Jan. 10, 2003

Related U.S. Application Data
(60) Provisional application No. 60/347,706, filed on Jan. 10, 2002.
(60) Provisional application No. 60/360,362, filed on Feb. 28, 2002.

(51) Int. Cl. A61K 38/21; A61K 31/739

(52) U.S. Cl. 424/85.5; 514/54

(57) ABSTRACT

The volume of an intracranial glioma tumor is decreased by contacting the tumor with both LPS and IFNγ. This protocol resulted in the eradication of detectable tumors in animal subjects.

### CROSS REFERENCE TO RELATED APPLICATION

This application claims priority from U.S. provisional patent application serial No. 60/347,706, filed Jan. 10, 2002 and U.S. provisional patent application No. 60/360,362, filed Feb. 28, 2002.

### FIELD OF THE INVENTION

The invention relates generally to the fields of medicine, neurology, and oncology. More particularly, the invention relates to compositions and methods for treating intracranial tumors.

### BACKGROUND

A number of clinically important intracranial tumors are known. Primary tumors such as astrocytomas, glioblastomas, oligodendrogliomas, and ependymomas originate from normal brain tissue. Intracranial tumors can also arise from other issues such as the meninges (meningiomas), the pituitary or pineal glands, or nerve tissue found at the base of the brain (e.g., acoustic neuromas or schwannomas). Secondary intracranial tumors are those that originate from tissue outside the cranium, but migrate to a site within the cranium (e.g., a metastatic brain tumor).

Conventional therapies employed to treat intracranial tumors include surgical resection, radiotherapy, and chemotherapy. Two or more of these are often used in combination. These conventional therapies each suffer well known drawbacks and are often only marginally effective for treating aggressive tumors such as glioblastoma multiforme. Major drawbacks of surgical resection relate to tumor accessibility. Attempts to remove tumors surrounded or intercalated in healthy brain tissue often cause injury to the healthy tissue. In many cases, surgical removal of the entire tumor is not practicable. In such cases, any non-removed portions of the tumor can continue to grow. Tumor inaccessibility is a particular problem for tumors such as glioblastoma multiforme that invade many different areas of healthy tissue.

Both radiotherapy and chemotherapy of intracranial tumors are often less successful than desired for similar reasons. In each method, the dose required to kill the tumor cells also results in the killing of healthy cells. The side effects from these treatments can therefore be prohibitively severe. An additional complication with chemotherapy is that the blood-brain barrier often creates drug delivery problems.

Given the drawbacks and lack of efficacy of conventional treatments, much research has been devoted to finding more effective and less injurious methods for treating intracranial tumors.

### SUMMARY

The invention relates to the discovery of an effective treatment for intracranial tumors. The treatment involves contacting the tumor in situ with a cocktail including a microglial cell activator such as a cocktail containing both lipopolysaccharide (LPS) and gamma interferon (IFNγ). Intratumoral injection of a LPS/IFNγ cocktail resulted in vigorous microglial cell activation accompanied by a rapid and dramatic regression of the tumor in several animal subjects. In some cases, the tumor was eradicated beyond detection.

Accordingly, the invention features a method for reducing the volume of a tumor (e.g., a glioma) contained within the cranium of a subject. The method includes the step of contacting the tumor with a purified microglial cell activator in an amount effective to reduce the volume of the tumor. In preferred variations of the invention, the purified microglial cell activator includes a purified LPS and/or a purified IFNγ. The step of contacting the tumor can be performed by injecting the purified microglial cell activator into the tumor or by placing into the brain of the subject a substrate (e.g., one made of a polymer matrix (e.g., DL-lactic-co-glyolic acid), gelatin sponge or gauze, collagen sponge, polydioxanone, porous polyethylene, cellulose gauze, or a starch matrix) containing the purified microglial cell activator. A variation of the foregoing method includes the additional step of surgically removing a portion of the tumor from the subject.

Also within the invention is a composition for reducing the volume of a tumor contained within the cranium of a subject. The composition includes a purified LPS, a purified IFNγ, and a pharmaceutically acceptable carrier.

The invention further features a kit for reducing the volume of a tumor contained within the cranium of a subject. The kit includes a purified microglial cell activator and printed instructions for using the purified microglial cell activator to reduce the volume of the tumor contained within the cranium of a subject.

In another aspect, the invention includes a method for activating a microglial cell contained within the cranium of a subject. This method includes the step of contacting the cell with a purified microglial cell activator in an amount effective to activate the cell.

Unless otherwise defined, all technical terms used herein have the same meaning as commonly understood by one of ordinary skill in the art to which this invention belongs.

Use of the term “purified” with respect to compounds and proteins, refers to compounds and proteins that are substantially separated (e.g., 10, 20, 30, 40, 50, 60, 70, 80, 90, 95, 97, 98, 99, or more % free from contaminants as measured by high performance liquid chromatography (HPLC) or a similar method) from other compounds and proteins that are present in a cell or organism in which the compound or protein occurs. Compounds or proteins made or processed by methods involving a step performed by the voluntary action of a human being are also considered “purified”. For example, a purified LPS is an LPS that has been purified from a bacterium. Similarly, a purified IFN-γ is IFN-γ that has been purified from a eukaryotic or bacterial cell.

Although methods and materials similar or equivalent to those described herein can be used in the practice or testing of the present invention, suitable methods and materials are described below. All publications, patent applications, patents, and other references mentioned herein are incorporated by reference in their, entirety. The particular embodiments discussed below are illustrative only and not intended to be limiting.

### BRIEF DESCRIPTION OF THE DRAWINGS

The above and further advantages of this invention may be better understood by referring to the following description taken in conjunction with the accompanying drawing, in which:

FIG. 1 is a graph of changes in tumor volumes as determined by magnetic resonance (MR) imaging of the animal subject over time. Times of treatment are indicated by arrows.

### DETAILED DESCRIPTION

The invention provides methods and compositions for treating intracranial tumors and for activating microglial cells in situ. The below described preferred embodiments illustrate adaptations of these methods and compositions. Nonetheless, from the description of these embodiments, other aspects of the invention can be made and/or practiced based on the description provided below.

### Treating an Intracranial Tumor

The invention provides a method for reducing the volume of a tumor contained within the cranium of a subject. The method includes the step of contacting the tumor with a purified microglial cell activator in an amount effective to reduce the volume of the tumor. Any suitable method for delivering a substance to a site in the cranium of a subject may be used to contact the tumor with the purified microglial cell activator. For example, the purified microglial cell activator can be delivered to an intracranial tumor in a subject by opening the skull (e.g., by drilling a burr hole) to expose the interior of the cranium, and then injecting the activator into or onto the tumor. As another example, after exposing the interior of the cranium and making the tumor site accessible, a substrate containing the purified microglial cell activator can be placed in the tumor site. Where all or a portion of a tumor has been surgically resected, the substrate can be placed into the cavity formed by removal of the tumor. The substrate can be any material suitable for this purpose. Examples of suitable materials include polymer matrices (e.g., DL-lactic-co-glyolic acid), gelatin sponges and gauzes, collagen sponges, polydioxanone, porous polyethylene, cellulose gauze, and starch matrices.

### Intracranial Tumors

The invention is based on the discovery that intracranial administration of an appropriate microglial cell activator can cause the regression of an intracranial tumor in a subject. In the experiments described herein, injection of LPS/IFNγ into intracranial glioma tumors led to killing of the tumor cells and, in some cases, the eventual eradication of the tumor. The invention is thus thought to be particularly effective for treating glial tumors. Nonetheless, the methods and compositions described herein are believed suitable for killing many different types of tumors located in a cranium, including various primary tumors and secondary tumors. A number of these are described below.

Primary intracranial tumors are tumors originating from intracranial tissue. Primary tumors derived from brain are classified by their histological and cytological features that resemble elements of the nervous system (see McKeever et al., In: Garcia J H, Budka H, McKeever P E, Sarnat H B, Sima A A F, eds. Neuropathology: The Diagnostic Approach. Philadelphia, Mosby 31-95, 1997). For example, gliomas are tumors with glial features, neuronal tumors resemble neurons, and neuroembryonal tumors resemble parts of the developing brain. Various forms of cancerous brain tumors include medulloblastomas, brain lymphomas, neuroectodermal tumors, neurocytomas, neuroepithelial tumors, and gliomas. Tumors of mixed lineage include anaplastic oligoastrocytomas (E. C. Burton and M. D. Prados, Curr. Treat. Options Oncol. 1:459-468, 2000). Primary tumors of the meninges are meningiomas.

Secondary intracranial tumors are those arising from metastases. Cancerous cells from tumors outside the cranium may migrate to sites within the brain causing the formation of an additional tumor. These too are expected to be susceptible to the treatments described herein.

Based on the experiments described herein, the invention is thought to be particularly well-suited for treating gliomas. Gliomas constitute the majority of primary tumors originating in the central nervous system. The most frequently encountered of these tumors in adults are high-grade or malignant neoplasms of astrocytic and oligodendrocytic lineage, i.e., anaplastic astrocytoma, glioblastoma multiforme, and anaplastic oligodendroglioma, respectively. Low-grade gliomas are a diverse group of neoplasms including astrocytomas (low-grade astrocytoma), oligodendrogliomas and mixed oligo-astrocytomas (Walker, D. G., and Kaye, A. H., Australas Radiol. 45:472-482, 2001, V. W. Stieber Curr. Treat. Options Oncol. 2:495-506, 2001). Because of its aggressiveness and the lack of effective treatments, of the different primary brain tumors, glioblastoma multiforme is usually considered the most devastating.

In the Examples section below, intratumoral injection of a microglial cell activator was shown to be a potent anti-tumor protocol in a rat model of glioblastoma multiforme. In this model, RG2 cells are injected into the brain of rats. The tumors that become established resemble glioblastoma multiforme (e.g., high level of vascularization, aggressive invasiveness, and non-immunogeneic nature). See, Aas et al., J. Neurooncol. 23:175, 1995; Barth, J. Neurooncol. 36:91, 1998. The methods and compositions of the invention are therefore thought to be particularly useful for treatment of glioblastoma multiforme patients.

Although the invention is focused on the treatment of intracranial tumors, because the methods and compositions described herein might be used to treat tumors located outside the cranium, the invention also encompasses treatment of extracranial tumors.

### Subjects

Because subjects from many different species have microglia and are susceptible to acquiring an intracranial tumor, the invention is believed to be compatible with many types of animal subjects. A non-exhaustive exemplary list of such animals includes mammals such as mice, rats, rabbits, goats, sheep, pigs, horses, cattle, dogs, cats, and primates such as monkeys, apes, and human beings. Those animal subjects known to suffer from an intracranial tumor are preferred for use in the invention. In particular, human patients suffering from an intracranial or other tumor are suitable animal subjects for use in the invention. In the experiments described herein, the subject used were rats. Nonetheless, by adapting the methods taught herein to other methods known in medicine or veterinary science (e.g., adjusting doses of administered substances according to the weight of the subject animal), the compositions utilized in the invention can be readily optimized for use in other animals.

### Use of a Microglial Cell Activator in Conjunction with Other Therapies

While the method for reducing the volume of a brain tumor contained within the cranium of a subject can be performed by simply contacting the tumor with a microglial cell activator, this does not preclude the use of other methods for primary or adjunctive treatment. Thus, for example, a subject (e.g., a human patient) with a brain tumor might first have a portion of the tumor surgically removed prior to the step of contacting the remaining tumor (or tissue surrounding the tumor site) with a microglial cell activator. Intracranial surgical techniques are described in, e.g., Greenberg, M., Handbook of Neurosurgery 5th Ed., Thieme Medical Pub., 2000; Lindsay K. and I. Bone, Neurology and Neurosurgery Illustrated 3rd Ed., Churchill Livingstone, 1997. Radiotherapy, chemotherapy, or other less conventional therapies might also be used in conjunction with the microglial cell activator.

### Activating Microglial Cells In Situ

The invention also provides a method for activating a microglial cell contained within the cranium of a subject. This method includes the step of contacting the cell with a purified microglial cell activator in an amount effective to activate the cell. As with the method for reducing the volume of an intracranial tumor described above, the cell may be contacted with the microglial cell activator by any known method, e.g., intracranial injection or placement of a substrate containing the activator into the brain of a subject.

### Microglial Cell Activators

Activators of microglial cells include any molecules (e.g., polypeptides, nucleic acids, etc.) that induce or help induce an activated phenotype in a microglial cell. Activated microglia can be distinguished from unactivated microglia in a number of ways. Morphologically, activated microglia are larger, more rounded (fewer cell processes), and more mitotically active than unactivated microglia. Activated microglia also express different cell surface molecules than do unactivated microglia. For example, activated, but not unactivated microglia express MHC class II molecules, CR3/43, and a marker called MOMA-2. Activated but not unactivated microglia bind PK11195. The induction of cytokine/chemokine (e.g., TNFalpha, NO, prostaglandinE₂) production occurs as microglia become activated. Functionally, activated microglia manifest higher levels of target cell killing than their unactivated counterparts.

Examples of activators of microglial cells include LPS and IFN-γ. Although LPS derived from E. coli serotype 055:B5 was used in the experiments described in the Examples below, LPS dervied from other gram-negative species (e.g., Klebsiella pnemoniae, Psuedomonas aeruginosa, Salmonella spp., etc.) or strains that have similar activity might be used Although rat IFN-γ use is described in the Examples below, IFN-γ from other species or variants of IFN-γ (naturally occurring or engineered mutants) that have similar activity might be used.

In addition to LPS and IFN-γ, the invention also contemplates using other substances capable of activating microglia as well as macrophages. A number of such substances are known including derivatives of various microorganisms, antibodies as well as other synthetic ligands, and cytokines. In addition to LPS, bacterial derivatives known to activate macrophages and microglia include murein/peptidoglycan. Mycoplasma derivatives (especially membrane proteins from Mycoplasma fermetans) and yeast cell wall derivatives (e.g., Zymosan particles [see Fitch et al., J. Neuroscience 19:8182-8198, 1999]; and purfied beta 1.3/1.6 glucan) are expected to be useful for activating microglia. A macrophage-stimulating cytokine such as GM-CSF or M-CSF might be used in the invention. An example of an antibody that can activate macrophages, and perhaps microglia, is anti-RMA from BD Pharmingen (San Diego, Calif.). In addition, beta amyloid protein might be used.

A microglial cell activator may be just one of the foregoing substances or it can be a mixture of two or more such substances. For example, the experiments described below show that a cocktail of LPS and IFN-γ is effective at activating microglia and causing the killing of tumor cells.

### Effective Amounts

The microglial activators described above are preferably administered to a mammal (e.g., human) in an effective amount, that is, an amount capable of producing a desirable result in a treated mammal (e.g., reducing the volume of an intracranial tumor in a subject, or activating microglia in the brain of a subject). Such a therapeutically effective amount can be determined as described below.

Toxicity and therapeutic efficacy of the compositions utilized in methods of the invention can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one animal depends on many factors, including the subject's size, body surface area, age, the particular composition to be administered, time and route of administration, general health, and other drugs being administered concurrently. It is expected that an appropriate dosage for intratumoral administration of LPS would be in the range of about 0.0001-0.01 mg/kg body weight. An appropriate dosage for intratumoral administration of IFNγ is expected be in the range of about 50-500 Units/kg body weight.

### Pharmaceutical Compositions and Administration to a Subject

The compositions described above may be administered to animals including human beings in any suitable formulation. For example, a microglial cell activator may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal target site. Other methods of delivery, e.g., liposomal delivery or diffusion from a device impregnated with the composition, are known in the art. The compositions may be administered in a single bolus or multiple injections. In cases where a tumor is not eradicated by a first dose, the administration of additional doses is preferred. For parenteral administration, the compositions are preferably formulated in a sterilized form.

Generally, compositions used in methods of the invention are formulated into a pharmaceutical composition that is administered by direct injection into the tumor to be treated, or administered into the tumor bed subsequent to tumor resection. The compositions may be precisely delivered into tumor sites, e.g., into gliomas or other intracranial tumors, by using stereotactic microinjection techniques. For example, a subject to be treated can be placed within a stereotactic frame base that is MRI-compatible and then imaged using high resolution MRI to determine the three-dimensional positioning of the particular tumor being treated. According to this technique, the MRI images are then transferred to a computer having the appropriate stereotactic software, and a number of images are used to determine a target site and trajectory for microinjection. Using such software, the trajectory is translated into three-dimensional coordinates appropriate for the stereotactic frame. For intracranial delivery, the skull is exposed, burr holes are drilled above the entry site, and the stereotactic apparatus positioned with the needle implanted at a predetermined depth. Tumor resection operations may be carried out prior to positioning of the stereotactic apparatus, if desired. The composition can then be microinjected at the selected target site(s).

### Kits

The invention also provides a kit for reducing the volume of a tumor contained within the cranium of a subject. The kit of the invention includes a purified microglial cell activator; and printed instructions for using the microglial cell activator to reduce the volume of tumor in a subject. In preferred versions of the kit, the microglial cell activator includes purified LPS and/or purified IFNγ.

### EXAMPLES

### Example 1

### TNF-α and Nitric Oxide Production by Rat Microglia

The ability of cultured rat microglia to produce the soluble cytotoxic agents, TNF-αand nitric oxide, was examined. Rat microglia were harvested and plated in a 96-well plate at a density of 5×10⁵ cells/well in 200 μl of serum-free CellGro complete medium (MediaTech, Inc.). In some wells, the medium was supplemented with different concentrations of LPS alone (from E. coli serotype 055:B55; Sigma) while others were supplemented with a combination of LPS and recombinant rat IFN-γ (100 U/ml; R&D Systems). After 24 hours of incubation at 37° C., TNF-α protein in the supernatant of each well was quantified using a Cytoscreen rat-specific TNF-α ELISA kit (Biosource Intl., Camarillo, Calif.), and NO (nitrite) levels in the supernatant of each well were determined using the Griess reaction. All experiments were carried out in triplicate. The results showed that treatment of the cells with the combination of LPS/IFN-γ induced much greater production of both TNF-α and nitric oxide than that induced by LPS alone.

### Example 2

### Microglia Cytotoxicity on RG2 Glioma Cells

Co-cultures of microglia and RG2 cells were established and cytotoxicity assays were carried out as follows: isolated rat microglial cells (5×10⁵ cells/well) were added to a 96-well plate containing 200 μl/well of complete medium. After allowing the cells to adhere for one hour, the medium was aspirated and the appropriate number of RG2 cells (from 2.5×10⁴ to 1×10⁶ to yield effector:target [E:T] ratios from 0.5:1 to 20:1) was added to the wells in 200 μl of fresh, complete medium. As controls, glioma cells and microglia were cultured separately in other wells of the plate. Microglia-mediated tumor cytotoxicity was measured using a modified MTT reduction assay.

All experiments were performed in quadruplicate. Some co-culture experiments were carried out on glass cover slips to provide visual documentation of cytotoxicity. The cover slips were immersed in paraformaldehyde solution and then subjected to cresyl violet staining. The results of these cytotoxicity studies demonstrated that LPS/IFN-γ stimulated (as described in Example 1) microglia showed tumor cytotoxicity when co-cultured with RG2 glioma cells. This cytotoxicity increased with increasing E:T ratios.

To determine the contributions of TNF-α and NO to the tumoricidal activity of microglia, MTT cytotoxicity assays were carried out in the presence of TNF-α binding protein (TNF-bp; an inhibitor of TNF-α) and N^{G}-monomethyl-L-arginine (L-NMA; an inhibitor of NO production). In the presence of neutralizing levels of TNF-bp (100 μg/ml), the killing of co-cultured RG2 cells by LPS/IFN-μ stimulated microglia (E:T=10:1) was inhibited to levels below those exhibited by unstimulated controls. Likewise, L-NMA exposure significantly inhibited killing of co-cultured RG2 by LPS/IFN-γ stimulated microglia.

The effects of TGF-β on microglial tumor cytotoxicity were also investigated in MTT cytotoxicity assays in the presence of TGF-β. The results showed that TGF-β caused a dose-dependent decrease in microglial cytotoxicity. Moreover, TGF-β also caused concurrent decreases in microglial production of TNF-α and NO.

### Example 3

### TNF-α and TGF-β Expression in RG2 Gliomas

The expression of TNF-α and TGF-β mRNAs in RG2 gliomas was examined by Northern blotting. The results showed that RG2 tumors express high levels of TGF-β mRNA, but a lack of TNF-α mRNA expression. LPS/IFN-γ stimulated microglia showed expression of TNF-α mRNA as well as TGF-β mRNA. In in situ hybridization experiments, high TGF-β mRNA expression localized specifically to the glioma portion of tissue sections. ELISA assays also showed that cultured RG2 glioma cells produce substantially higher amounts of TGF-β protein than do cultured microglial cells.

### Example 4

### Intratumoral Injections of LPS/IFN-γ

RG2 tumors were induced in six rats by intrastriatal RG2 cell inoculation as previously described (Morioka et al. Glia 6:75-79, 1992; Morioka et al., Acta. Neuropathol. 83:590-597, 1992; Morioka et al., Neurosurgery 30:891-896, 1992). The tumors were allowed to grow for two weeks (a time when tumors typically reach about 3-4 mm in diameter). Three of the animals received intratumoral injections of LPS (5 μg/5 μl; E. coli serotype 055:B5) and rat IFN-γ (250 U/5 μl), diluted in 0.9% sterile saline, using the same burr hole as for the RG2 cell injections. Control animals received saline only. After four days, the animals were euthanized with an overdose of pentobarbital, perfused with Bouin's fixative (72.5% picric acid, 22.5% formalin, 5% glacial acetic acid), and processed for paraffin embedding and sectioning. Lectin staining of microglia using the B4-isolectin from Griffonia simplicifolia was carried out as previously described (Streit and Kreutzberg, J. Neurocytol. 16:249-260, 1987; Streit, J. Histochem. Cytochem. 38:1683-1686, 1990).

Light microscopic examination of sections of saline-injected RG2 gliomas revealed the presence of numerous microglial cells in and around the glioma. Sections of RG2 gliomas that received an intratumoral injection of LPS/IFN-γ showed a markedly enhanced microglial response characterized by a notable increase in the number of lectin-positive (microglial) cells, especially in association with areas of necrosis. In addition to being more numerous, lectin-reactive microglia in LPS/IFN-γ-treated tumors also stained more intensely and displayed a more activated morphology (i.e., they were rounded and hypertrophic). All LPS/IFN-γ-treated gliomas exhibited large central regions of necrosis that contained small surviving tumor cell islands which frequently contained a central blood vessel. In situ hybridization histochemistry showed that the islands of surviving tumor cells expressed high levels of TGF-β mRNA. The effects of LPS/IFN-γ treatment were also evident in microglial cells populating the peritumoral, normal brain tissue of the ipsilateral hemisphere. These cells often displayed a rounded morphology indicative of activation.

### Example 5

### Tumor Analysis

As described above, intracerebral glioblastomas were induced in three rats (BT1, BT2, BT3). BT1 and BT2 developed very large tumors within 12 days and were sacrificed because they showed behavioral abnormalities. The tumor grew more slowly in BT3, although after 22 days a large tumor that extended from the striatum caudally to the thalamus and lateral ventricle was observed. Tumor volume was estimated by MRI by manually outlining the regions of interest for each slice through the lesion, and then interpolating across the slices (accounting for the interslice gap). See, Bui et al., NMR imaging of the neuroprotective effects of estrogen. International Society for Magnetic Resonance in Medicine 7th Annual Meeting, Philadelphia, May 1999; Shi et al., Stroke 32:987-992, 2001.

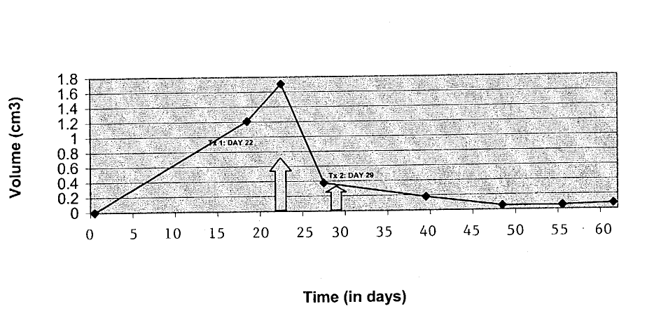

The tumor in BT3 was injected using the same burr hole and stereotactic coordinates (5 mm depth) with a cocktail containing 4 μg LPS and 400 units IFN-γ dissolved in 8 μl PBS. No abnormalities were noted in the subsequent 24 hour post-operative period. FIG. 1 shows the changes in tumor volume over a sixty day period.

MRI five days after treatment showed a substantial decrease in tumor noted. On day 30 after tumor induction, BT3 was intratumorally injected with a second round of the LPS/IFN-γ cocktail. Subsequent MRI showed that the tumor volume continued to decrease. At 71 days post-inoculation with glioma cells, the tumor was not detectable by MRI. BT3 was euthanized for histological study 86 days post-inoculation with glioma cells. No behavioral abnormalities were observed in BT3 throughout the study.

### Example 6

### Histology

After being euthanized, BT3 was perfused with 4% paraformaldehye and the brain was sectioned on a vibratome. Serial sections through the tumor site were collected and stained with cresyl violet and with anti-GFAP for astrocytes and GSA I-B₄ isolectin for microglia. Microscopic examination revealed that no tumor tissue remained. In place of the tumor, a large cavity surrounded by a dense astroglial scar was observed. Cresyl violet staining indicated extensive inflammatory cell infiltrates within the large cavity and perivascular inflammation in tissue surrounding the cavity. Large numbers of foamy macrophages containing intracytoplasmic phagosomes were found within the cavity. Macrophages were lectin-positive and scattered among remaining blood vessels. GFAP immunohistochemistry showed a dense glial scar surrounding the cavity. Activated, rounded microglia were seen with lectin staining in the parenchyma surrounding the cavity.

### Example 7

### Intratumoral Therapy

In additional experiments, RG2 tumor induction and intratumoral injections of LPS and rat IFN-γ were performed as described in Example 4 in three animals (W1, W2, and W3).

W1. One week after tumor cell implantation, MRI showed a small tumor beginning to grow in the right hemisphere of W1. Three weeks after implantation, a large tumor was present. A single injection of the LPS/IFN-γ cocktail was delivered intratumorally at this time. Six days later, MRI showed a dramatic reduction in tumor size. MRI at 36 days and 43 days post-implantation showed that the tumor was completely eradicated.

W2. One week after tumor cell implantation, no tumor was apparent by MRI in W2. Three weeks after implantation, however, a large, infiltrating tumor was observed. An intratumoral injection of LPS/IFN-γ was performed at this time. Six days later, the tumor appeared larger in size in coronal section, and a second intratumoral LPS/IFN-γ injection was delivered. Eight days after the second treatment, MRI showed dramatic regression of the glioma.

W3. One week after tumor cell implantation, no tumor was apparent by MRI in W3. Three weeks after implantation, however, a large, infiltrating tumor was observed. An intratumoral injection of LPS/IFN-γ was performed at this time. Six days later, the tumor had regressed considerably leaving a sizable cavity. Twenty-eight days post-implantation, a second intratumoral LPS/IFN-γ injection was delivered. At 36 days post implantation, the tumor was eradicated.

### Other Embodiments

It is to be understood that while the invention has been described in conjunction with the detailed description thereof, the foregoing description is intended to illustrate and not limit the scope of the invention, which is defined by the scope of the appended claims. Other aspect, advantages, and modifications are within the scope of the following claims.

What is claimed is:

1. A method for reducing the volume of a tumor contained within the cranium of a subject, the method comprising the step of contacting the tumor with a purified microglial cell activator in an amount effective to reduce the volume of the tumor.

2. The method of claim 1, wherein the purified microglial cell activator comprises at least one of a purified LPS and a purified IFNγ.

3. The method of claim 2, wherein the purified microglial cell activator comprises both a purified LPS and a purified IFNγ.

4. The method of claim 1, wherein the step of contacting the tumor comprises injecting the purified microglial cell activator into the tumor.

5. The method of claim 4, wherein the purified microglial cell activator comprises at least one of a purified LPS and a purified IFNγ.

6. The method of claim 5, wherein the purified microglial cell activator comprises both a purified LPS and a purified IFNγ.

7. The method of claim 1, wherein the step of contacting the tumor comprises placing into the brain of the subject a substrate comprising the purified microglial cell activator.

8. The method of claim 7, wherein the purified microglial cell activator comprises at least one of a purified LPS and a purified IFNγ.

9. The method of claim 8, wherein the purified microglial cell activator comprises both a purified LPS and a purified IFNγ.

10. The method of claim 7, wherein the substrate is a material selected from the group consisting of: a polymer matrix, gelatin, collagen, polydioxanone, porous polyethylene, cellulose, and starch.

11. The method of claim 1, wherein the tumor comprises a glioma cell.

12. The method of claim 1, further comprising the step of surgically removing a portion of the tumor from the subject.

13. A method for activating a microglial cell contained within the cranium of a subject, the method comprising the step of contacting the cell with a purified microglial cell activator in an amount effective to activate the cell.

14. The method of claim 13, wherein the purified microglial cell activator comprises at least one of a purified LPS and a purified IFNγ.

15. The method of claim 14, wherein the purified microglial cell activator comprises both a purified LPS and a purified IFNγ.

16. A method for reducing the volume of a glioma contained within the cranium of a subject, the method comprising the step of contacting the glioma with a purified LPS and a purified IFNγ in an amount effective to reduce the volume of the glioma.

17. A composition for reducing the volume of a tumor contained within the cranium of a subject, the composition comprising a purified LPS, a purified IFNγ, and a pharmaceutically acceptable carrier.

18. A kit for reducing the volume of a tumor contained within the cranium of a subject, the kit comprising:
(A) a purified microglial cell activator; and
(B) printed instructions for using the purified microglial cell activator to reduce the volume of the tumor contained within the cranium of a subject.

19. The kit of claim 18, wherein the purified microglial cell activator comprises at least one of a purified LPS and a purified IFNγ.

20. The method of claim 19, wherein the purified microglial cell activator comprises both a purified LPS and a purified IFNγ.
